(19) United States
(12) Patent Application Publication
(10) Pub. No.: US 2009/0239251 A1
(43) Pub. Date: Sep. 24, 2009

(54) Method for Detecting Nanoparticles and the Use Thereof

(75) Inventors: Emmanuel Fort, Paris (FR); Christian Joel Claude Ricolleau, Paris (FR); Sandrine Patricia Leveque-Fort, Paris (FR); Eric Ronan Le Moal, Saint-Cyr-L'ecole (FR)

Correspondence Address: HARNESS, DICKEY & PIERCE, P.L.C., P.O. BOX 828, BLOOMFIELD HILLS, MI, 48303, US

(73) Assignee: CENTRE NATIONAL DE LA RECHERCHE SCIENTIFIQUE(CNRS), Paris (FR)

(21) Appl. No.: 12/084,683

(22) PCT Filed: Nov. 9, 2006
(86) PCT No.: PCT/EP2006/068316

§ 371 Date: May 7, 2008

(30) Foreign Application Priority Data
Nov. 9, 2005 (FR) 0511384

(51) Int. Cl. C12Q 1/02; G01N 21/00; G01N 33/00; G01N 33/68; G01N 21/55; G01N 21/64

(52) U.S. Cl. 435/29; 436/164; 436/94; 436/86; 436/87; 422/82.05; 356/445; 250/459.1; 977/755; 977/777

(57) ABSTRACT

The invention relates to a method for detecting and/or qualifying nanoparticles whose mean size is less than 60 nm, which exhibit a plasmon resonance and are located on the top surface of a flat solid support. A device for carrying out the inventive method and the use thereof are also disclosed.

The aim of the invention is a method for detection and/or quantification of nanoparticles of a size less than 60 nm having a plasmon resonance and present on the upper surface of a flat solid support. The invention also comprises a device for executing such a method and its applications.

In numerous fields related to biology experimental work entails the observation of molecules using fluorescence measures. Particular examples are studies on the reactional dynamics of drugs or biological molecules playing an essential role in the function of a cell (proteins, RNA, . . . ). The veritable issue of these studies is obtaining this type of information at the level of a single molecule. The activity of a drug at the level of the genome of a cell can, for example, bring into play the action of a single molecule to then be observed and characterised. The weakness of the fluorescence signal is then a major limitation.

On the other hand, fluorescent marking presents another major limitation associated with photodestruction processes of markers. Fluorescent molecules can absorb and emit only a limited number of photons before being destroyed, and so the average fluorescence of a marked sample diminishes over time. This phenomenon limits the duration over which a marked molecule can be tracked. In addition, the degradation of fluorescent markers makes the long-term archiving impossible: for example, a biochip becomes illegible a few weeks after its manufacture.

The problem with photodestruction of fluorescent markers has been partially resolved due to recent progress made in the use of semi-conductive nanocrystals such as fluorophores. However, these markers more resistant to photodestruction introduce novel problems, such as that of flickering and biocompatibility, which limit their efficacy in dynamic studies in a biological medium.

There are numerous types of non-fluorescent markers, especially metallic nanoparticles, which have unique optical properties. In particular, they exhibit, for certain wavelengths, marked absorption resonances associated with the collective oscillations of their conduction electrons. Nanoparticles are accordingly being used more and more as tracers in molecular biology by replacing fluorescent markers. They are not photodestroyed, allow multicolour marking and emit a signal much more intense than standard fluorophores. Their detection is based on their capacity to diffuse light (Rayleigh diffusion). But, this aptitude diminishes with their size and in practice nanoparticles of a size less than 30 nm cannot be detected. This size limit is prohibitive for any dynamic study, in particular for tracking biomolecules in vivo, as the nanoparticles would for example make it impossible for marked biomolecules to migrate across membranal canals.

As a consequence, marking by nanoparticles is often done or modified a posteriori (in the case of biomarker especially). For example, when smaller nanoparticles are used for marking, it is necessary to increase their size a posteriori, so as to then increase the light signal and be able to detect them (silver amplification).

Recent techniques incorporating photothermal effect measures enable detection of nanoparticles under 10 nm. These techniques, which are not yet available commercially, do not offer full-field imaging, just point by point. Dynamic studies thus remain difficult, or even impossible due to the slow pace of measurements. In addition, these indirect techniques require the use of two light sources.

Nanoparticles are also used in observation devices for total reflection attenuation (TRA) on a metallised slide. With considerable sensitivity this technique measures the variations in refraction indices near a surface. The surface plasmon mode by a metallic film deposited on a transparent support can be excited from lighting the film via the support, at a highly precise angle. Only at this angle is reflection of the light on the metal fully attenuated. Measuring this angle detects with considerable sensitivity the variations in indices near the surface in the sample studied. Those nanoparticles approaching the surface of the metallised lame locally amplify the index of the medium. This technique when used in imaging requires specific apparatus.

Finally, there are numerous non-optical techniques applied to biosensors (gravimetric, electrochemical and electric). All these types of detection are relatively marginal and require considerable and specific financial investment.

What remains is to find a method which is efficacious, reliable and easy to execute, allowing detection and/or quantification of nanoparticles whereof the size is less than or equal to 60 nm, especially less than or equal to 20 nm on the surface of a solid support. This is the very object of the present invention.

The inventors have disclosed that it was possible to detect nanoparticles of a few nanometers in diameter located at a distance less than a few nanometres from a metallic surface covering a flat solid transparent support. The inventors have especially been able to show that when a nanoparticle approaches a metallic surface evanescent components present in its near field couple with the surface plasmon of the metal, causing an energy transfer to the latter. This energy can then be transferred in the form of a cone of light emitted by the rear face of the thin metallic film (cf. FIG. 5). Using a classic optical microscope, it is thus possible to detect and image in full field nanoparticles of a few nanometers in diameter.

Therefore, in a first aspect, the aim of the invention is a method for detection and/or quantification of nanoparticles present on the upper surface of a flat solid support, said nanoparticles having a plasmon resonance, said solid support comprising a solid transparent support coated on its upper surface by a metallic film having a surface plasmon and the index of said transparent support being greater than that of the medium in which said nanoparticles are located, said method being characterised in that it comprises the following steps:
a) lighting by the upper or lower surface of said solid support to illuminate the nanoparticles optionally present on the upper surface;
b) detection and/or quantification of light originating from the nanoparticles by the lower surface.

In a preferred embodiment, the method for detection and/or quantification of nanoparticles according to the invention is characterised in that:
at step a) lighting is generated by the upper surface; and
at step b) detection and/or quantification of light originating from the nanoparticles by the lower surface is done by releasing the direct light originating from the lighting passing through the metallic film,

or in that:
at step a) lighting is generated by the lower surface; and
at step b) detection and/or quantification of light originating from the nanoparticles by the lower surface is done by releasing reflected light originating from the lighting.

The aim of lighting by the upper or lower surface of said support, to illuminate nanoparticles optionally present on the upper surface, in the method of the invention is to excite the nanoparticles at a wavelength related to the frequency of plasmon resonance of said nanoparticles.

“Light originating from nanoparticles by the lower surface” is understood here in particular to designate light originating from the nanoparticle at the same wavelength as the excitation wavelength of the nanoparticle and which is transmitted by the lower surface.

In an embodiment also preferred, the method for detection and/or quantification of nanoparticles according to the invention is characterised in that the nanoparticles are metallic nanoparticles, especially selected from nanoparticles of gold, silver, aluminium, platinum or copper, the nanoparticles of gold or silver being the most preferred.

In an also preferred embodiment, the method for detection and/or quantification of nanoparticles according to the invention is characterised in that the metallic film covering the upper surface of the solid transparent support is selected from a film whereof the metal is that of the nanoparticles to be detected.

This metallic film preferably has a thickness of between 5 nm and 500 nm, especially between 20 nm and 80 nm.

This metallic film has a thickness of between 20 nm and 80 nm in particular when the excitation wavelength of the nanoparticles is a wavelength of light located in the visible spectrum.

In an also preferred embodiment, the method for detection and/or quantification of nanoparticles according to the invention is characterised in that said nanoparticles likely to be present on the upper surface of the solid support to be detected and/or quantified are at a distance less than or equal to the excitation wavelength of said nanoparticles, preferably less than or equal to half of the excitation wavelength of said nanoparticles.

The nanoparticles likely to be present on the upper surface of the solid support to be detected and/or quantified are preferably at a distance less than or equal to 500 nm, in particular if the light used for the lighting is in the visible range.

The nanoparticles likely to be present on the upper surface of the solid support to be detected and/or quantified are preferably at a distance less than or equal to 500 nm, preferably less than or equal to 400 nm, 300 nm or 200 nm, of the upper surface of the solid support, a distance less than or equal to 200 nm being the most preferred.

According to a particular embodiment of the method according to the invention, the metallic film located at the upper surface of the support is covered with a transparent film allowing the minimum distance between the nanoparticles and the metallic film to be adjusted.

The metallic film located at the upper surface of the support is preferably covered with a transparent film constituted by non-conductive metal, especially selected from metallic oxides such as titanium oxide, aluminium oxide (alumina).

This transparent film preferably has a thickness less than or equal to 50 nm.

In an also preferred embodiment, the method for detection and/or quantification of nanoparticles according to the invention is characterised in that at step a) the lighting is generated by the upper surface. In this embodiment, at step b) the light originating from the nanoparticles is preferably transmitted to the lower surface via said support.

In an also preferred embodiment, the method for detection and/or quantification of nanoparticles according to the invention is characterised in that at step a) lighting by the upper or lower surface of said support is generated either:
with a white light source or with a polychromatic light whereof the excitation wavelengths contain at least one excitation wavelength related to the frequency of plasmon resonance of said nanoparticles; or
with a monochromatic light source whereof the excitation wavelength is related to the frequency of plasmon resonance of said nanoparticles.

According to an also preferred embodiment, the method for detection and/or quantification of nanoparticles according to the invention is characterised in that at step b) the detection and/or quantification of the light originating from the nanoparticles on the lower surface is done by means of a microscope, where necessary coupled to a CCD camera.

Such a microscope is especially a reflection microscope fitted with a digital large-aperture immersion lens and preferably equipped with a cache which, when lighting is generated by the upper surface, masks or eliminates direct light originating from the lighting passing through the metallic film, or which when lighting is generated by the lower surface masks or eliminates the reflected light originating from the lighting.

It should be noted that using a digital large-aperture immersion lens for collecting light originating from the nanoparticles on the lower surface and to be detected and/or quantified is also particularly preferred for configuration with lighting by the upper part (see example 4 B).

The microscope used for detection of nanoparticles can also be a “point by point scanning” microscope of confocal or scanner microscope type. A digital large-aperture immersion lens can be used to collect the cone of light, described in Example 2 and shown in FIG. 5, coming from the particles via the lower face. This microscope could in particular be equipped with a “parabolic” immersion lens based on a principle such as that described in FIG. 1 page 48 in the article by Dr. Thomas Ruckstuhl in the Journal “Biophotonics International” of September 2005.

According to an also preferred embodiment, the method for detection and/or quantification of nanoparticles according to the invention is characterised in that the nanoparticles to be detected and/or quantified have a diameter of less than or equal to 60 nm, preferably less than or equal to 40 nm, 30 nm or 20 nm, a diameter less than or equal to 20 nm being particularly preferred.

In a particular embodiment, the method for detection and/or quantification of nanoparticles according to the invention is characterised in that the nanoparticles to be detected and/or quantified have different colours associated with their plasmon resonance.

In a particular embodiment, the method for detection and/or quantification of nanoparticles according to the invention is characterised in that said support is a solid transparent support coated on its upper surface by a metallic film on which is fixed a probe compound capable of specifically recognising a target compound to be detected and/or quantified by means of or by the presence of nanoparticles.

Nanoparticles today are currently used in biology for the capture or the marking of compounds, especially biological, such as proteins, neurotransmitters, nucleic acids, lipids or even carbohydrates but also cells. In general, the surface of nanoparticles is coated with or functionalised by a compound capable of binding specifically to the target compound to form a complex (for example a complex formed by specific hybridation of complementary nucleic acids, complex of antibody-antigen type, ligand-receptor, etc.). The detection or quantification of these nanoparticles thus complexed on the target compound will be directly correlated to the presence and/or the quantity of the target compound present in a sample.

In a novel aspect, the aim of the present invention is therefore a method for detection and/or quantification of a target compound in a sample by means of a solid support, in which the detection and/or quantification of said target compound is correlated to detection and/or quantification of nanoparticles, characterised in that the nanoparticles are detected and/or quantified by a method according to the invention.

The invention also comprises a method for detection and/or quantification of nanoparticles according to the invention or a method for detection and/or quantification of a target compound in a sample according to the invention, characterised in that the nanoparticles to be detected and/or quantified are used as specific marker of said target compound to be detected and/or quantified.

In a preferred embodiment, the nanoparticles to be detected and/or quantified are coated with (or functionalised by means of) a compound capable of binding specifically to the target compound, especially a complementary nucleic acid of the target compound if the target compound is a nucleic acid, an antibody capable of specifically recognising the target compound if the target compound is a protein, a ligand, especially a neurotransmitter, capable of specifically recognising the target compound if the target compound is a receptor.

Accordingly, the present invention relates a method for detection and/or quantification of nanoparticles or a method for detection and/or quantification of a target compound in a sample according to the invention, characterised in that the probe compound or the target compound is selected from the group of compounds constituted by nucleic acids, polypeptides, nucleic acids peptides (PNA), lipopeptides, glycopeptides, neurotransmitters, carbohydrates, lipids, preferably nucleic acids, polypeptides, neurotransmitters or carbohydrates.

In a preferred embodiment of the methods according to the invention, said flat solid support is made of glass.

In yet another aspect, the present invention comprises a device for detection and/or quantification of nanoparticles present on the upper surface of a flat solid support, said nanoparticles having a plasmon resonance, said solid support comprising a solid transparent support coated on its upper surface by a metallic film having a surface plasmon and the index of said transparent support being greater than that of the medium in which said nanoparticles are located, said device comprising a light source allowing lighting of the upper surface or the lower surface of said support and a system for detection and/or quantification of the light transmitted by the lower surface of said support, characterised in that said device further comprises a system for eliminating or masking: either
reflected light originating from the light source when the lighting is generated by the lower surface of the solid support; or
direct light transmitted by the light source when the lighting is generated by the upper surface of the solid support.

The device according to the invention is preferably a reflection microscope, particularly fitted with an immersion lens, preferably again a digital large-aperture lens, such as for example a microscope used traditionally for total internal reflection fluorescence (TIRF), this microscope being characterised in that it is fitted with a cache which when lighting the nanoparticles is generated by the upper surface of the flat solid support, this cache masks or eliminates direct light originating from the lighting passing through the metallic film, or a cache which when the lighting is generated by the lower surface, masks or eliminates reflected light originating from the lighting.

More preferably, such a device is illustrated in FIG. 6.

The present invention also comprises in another aspect using a method or a device according to the present invention, for:
detection and/or quantification of compound present in a sample;
in vitro diagnosis of illness in a patient linked to the presence or concentration of a particular compound in a biological sample from said patient;
biological imaging of systems confined to a few tens of nm, especially for the study of membranal transfers, precise localisation of compound in a cellular compartment or biosensors, by replacing traditional techniques: total internal reflection fluorescence (TIRF) and surface plasmon resonance imaging (SPR); and
full-field imaging of nanoparticles, especially of diameter less than or equal to 20 nm, over a thickness less than or equal to 500 nm, preferably less than or equal to 200 nm.

This ultra-sensitive full-field imaging displays molecular interactions on the surfaces, representing a fundamental interest in cellular and molecular biology as numerous processes of molecular transport are trans-membranal. Examples are activation of cells by hormones, neurotransmitters and antigens, adhesion of cells to surfaces (in biofilms especially), electronic transport in membranes, dynamics of membrane and cytoskeleton; events linked to cellular secretions and fusion of vesicles with membranes. The extreme finesse of the probed zone by this technique detects only those nanoparticles attached to the surface. Those present in the surrounding medium are invisible with this technique.

The field of biosensors is also a considerable field of application for this invention.

This novel method opens the way for followup studies on single biomolecules, as well as on ultra-sensitive and rapid imaging of samples marked by means of nanoparticles of a few nanometers.

Nanoparticles today are used widely as media and as markers for detecting or amplifying protein-protein reactions, of antigen/antibody (immunological reagents) or ligand/receptor type, or between strands of complementary nucleic acids (probed DNA). There is currently a very wide range of nanoparticles commercially available whereof the size and surface properties (hydrophily, functional groups, . . . ) are extremely varied and can a priori be satisfactory for fixing biomolecules of interest by adsorption or covalence. A large quantity of protocols familiar to those skilled in the art is described in the literature for making such settings, whether on these nanoparticles or on the SPR supports used for their detection. These nanoparticles fitted with such a detection method according to the invention contribute novel solutions for manufacture and conducting of tests, as well as for constructing microsystems of “lab-on chips” type (DNA or protein chips).

The legends of the following figures and examples are intended to illustrate the invention without limiting its scope.

### LEGENDS OF FIGURES

FIG. 1: Diagrams representing a surface plasmon: EM field induced by loads, amplitude of the evanescent wave associated with the side of the metal and the side of the dielectric, theoretical cartography of the electric field Ez (W. L. Barnes et al., Nature 424 p. 824 (2003)).

FIG. 2: Dispersion relation of the surface plasmon (curve tending towards a horizontal asymptote shown in dots) for a metal/sample interface (of index n′), the straight line of greatest slope (dark grey straight line) represents the limited dispersion curve for a beam coming from the side of the sample with a raking incidence, the straight line of least slope (light grey straight line) shows that this limit is pushed back for beams originating from a higher index medium n>n′. Coupling with the plasmon metal sample is then possible. This is how the plasmon can generate a cone of light or be excited by the rear face.

ωₚ=plasma frequency

n=refraction index of the substrate (i.e. glass)

FIG. 3: Probability of energy transfer of a fluorophore at λ=600 nm as a function of its distance from the surface. Dotted line: average for a set of dipoles oriented randomly (W. H. Weber and C. F. Eagen, Opt. Lett. 4 (8) p. 236 (1979)) (“emission probability”).

FIG. 4: Power dissipated as a function of the wave vector parallel to the surface normal for a silver nanoparticle of radius 10 nm (a), 30 nm (b) and 50 nm (c), lit under normal incidence at λ=500 nm and located at 50 nm from the metallic interface (J. Soller and D. G. Hall J. Opt. Soc. Am. B 19 (5) p. 1195 (2002)).

FIG. 5: Cone of light originating from coupling with the surface plasmon: in this case the coupling originates from nanoroughness present at the surface of the thin metallic film (N. Fang, Opt. Express 11 p. 682 (2003)).

FIG. 6: Digital large-aperture immersion lens used traditionally for fluorescence imaging and in particular for total internal reflection imaging (TIRF) fitted with a cache for executing the method of the invention (lens image from the web site: http://www.olympusmicro.com/primer/).

FIG. 7: Simulation of the relative amplitude of the evanescent wave relative to that obtained in total internal reflection as a function of the angle of incidence for thicknesses of silver of 30, 40, 50 and 60 nm. The maxima are obtained between 40 and 50 nm.

FIG. 8: Binarised image revealing on a microscope slide the presence of individual nanoparticles of silver, 20 nm in diameter, deposited on a flat solid substrate covered with a film of silver 50 nm thick and with an amorphous layer of alumina 15 nm thick, after lighting of the upper part of the support with a 100 W halogen lamp.

### EXAMPLES

### Example 1

### Principle of Nanoparticle/Surface Plasmon Coupling

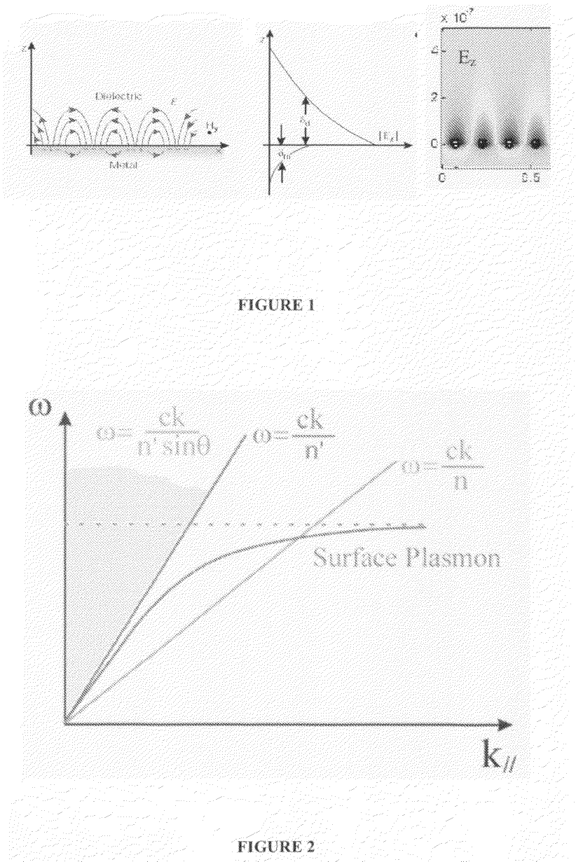

When a fluorophore approaches a metallic surface, a fresh energy transfer method appears via the surface plasmon. The surface plasmon is a collective oscillation mode of the conduction electrons at the interface between a metal and a dielectric. These oscillations generate an evanescent electromagnetic wave (EM) which spreads at the surface of the metal (cf. FIG. 1).

So there is coupling (i.e. energy transfer) between two modes, the energy and the components of the wave vector of these two modes must coincide. A beam of light arriving at the metallic surface, from the side of the sample associated with an index n′, and making an angle θ with the normal at the surface, has a linear dispersion relation: ω=c k/n′ sin θ where k=2π/λ is the norm of the wave vector and c is the speed of light in a vacuum. The graph shown in FIG. 2 represents all these dispersion relations as a function of the component of k parallel to the surface of the metal. Irrespective of the angle of incidence, the dispersion curve associated with the beam of incident light is located to the left of the straight line ω=c k/n′ associated with a raking incident wave. Since the dispersion curve of the surface plasmon is located to the right of this line, it never crosses the dispersion curves associated with the incident beams to the side of the sample. It is thus impossible to excite the plasmon with an incident EM wave on the surface of the side sample. Inversely, the plasmon cannot emit light from this side.

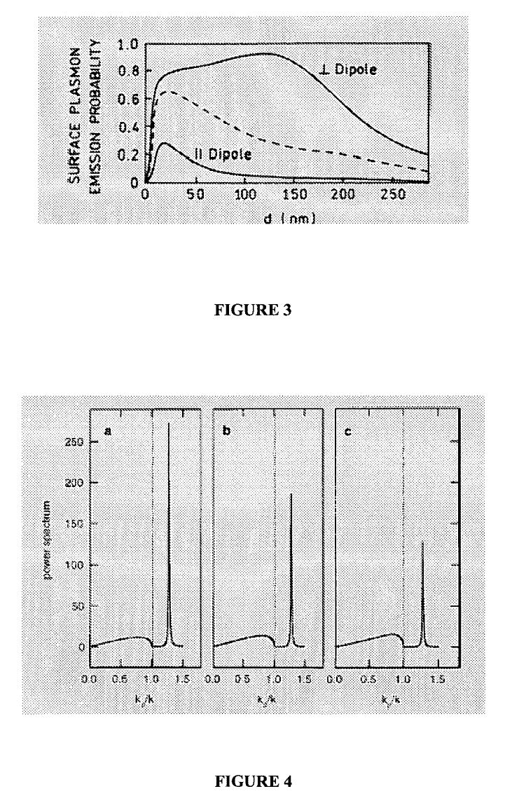

The fluorescence emitted by a fluorophore located far from the metallic surface (beyond the evanescent wave) cannot thus excite the plasmon. Only those evanescent components associated with the emission dipole and which are present only in the field near the fluorophore can couple with the plasmon (cf. FIG. 2). In fact, the evanescent components can have an arbitrary large component of the wave vector parallel to the surface, the orthogonal component becoming imaginary (i.e. evanescent). Therefore, only those fluorophores located in the evanescent wave, i.e. typically at less than 200 nm, can excite the surface plasmon. This coupling depends on the orientation of the dipole emitter of the fluorophore relative to the surface. It represents around 60% of the energy lost by a set of fluorophores oriented randomly (cf. FIG. 3) and can reach 93% when the dipole is oriented orthogonally to the surface.

Similarly as for a fluorophore, this effect is present in the case of a nanoparticle located near a metallic surface. The metallic nanoparticle behaves like a dipole.

FIG. 4 presents the results of modelling a silver nanoparticle located at 50 nm from a metallic interface, lit under normal incidence at λ=500 nm, for radii of 10, 30 and 50 nm. According to these theoretical calculations, the proportion of energy transmitted by the nanoparticle to the surface plasmon reaches 46% (for a radius of 10 nm). An even greater proportion is expected when the distance separating the nanoparticle from the surface metallic is reduced. The nanoparticle/surface plasmon coupling is all the stronger since the nanoparticles are small (cf. FIG. 4). In fact, for small nanoparticles, the distribution of the components of the EM field as a function of the wave vector is greater in major spatial frequencies. In other words, the relative proportion of the evanescent field (near field) is greater compared to that associated with the propagating field. Yet it is these very components which are likely to couple with the surface plasmon (peaks in FIG. 4).

The diffusion yield of the nanoparticles drops when their size decreases. This drop is caused by the relative decrease in the radiative processes of energy dissipation (Rayleigh diffusion) relative to the processes of internal energy dissipation (absorption). Small nanoparticles (typically under 10 nm in radius) are thus difficult to detect by diffusion. This diffusion corresponds in FIG. 4 to the standardised values of k under 1 (non-evanescent field). Current methods for detecting such small nanoparticles are methods for detection of the photothermal effect and are based on absorption (see international patent application published under number WO 2004/025278).

### Example 2

### Detection of Nanoparticles

Since surface plasmons cannot a priori couple to the propagative field, the energy transmitted to these non-radiative modes is lost (dissipated in the form of heat in the metallic film). By optimising the distance separating them the efficacious absorption section of the system formed by the nanoparticle and the metallic surface is increased. It is however possible to recover this energy in the form of light. Actually, if the index of the medium located on the rear face of the film is greater than that located on the front face and if the metallic film has a determined thickness, then the surface plasmon couples with the propagative EM field (cf. FIG. 2) by emitting a cone of light via the rear face (cf. FIG. 5).

The optimal thickness of the metallic film to enable this coupling (typically a few tens of nanometers) depends on the excitation wavelength and on the refraction index of the media on either side of the metallic film.

By making an image of the metallic surface via the transparent support it is thus possible to display nanoparticles of a few tens of nanometers in full field, with a standard microscope.

### Example 3

### Nanoparticles as Biological Markers

Metallic nanoparticles have a plasmon resonance, i.e. a frequency for which their efficacious absorption section becomes very large (relative to their geometric size). This phenomenon is due to the confinement of the oscillation mode of the conduction electrons. This resonance depends on the form, size and nature of the nanoparticles. It is very marked for silver or gold nanoparticles, for example. It is essential to accord the excitation wavelength of the nanoparticles on their frequency of plasmon resonance so as to detect them, this plasmon resonance frequency being especially a function of the nature, form and environment of these nanoparticles, in particular their distance relative to the metallic film. This property of nanoparticles allows multicolour marking (as for fluorescence) using several types of different nanoparticles (in nature or form, for example). Relative to fluorescent markers, nanoparticles have the advantage of not being photodestroyed.

The marking of biological cells by nanoparticles functionalised by a few nanometers is mastered today as it is widely used for electron microscopy observations. The method according to the invention does not therefore create a problem for the marking phase already commercially available.

### Example 4

### Excitation Configurations

Several modes of excitation are possible for exciting fluorophores:
A) Fluorophores can be excited in evanescent waves by exciting the plasmon.

Via the same method as coupling between the plasmon and light emission in the form of a cone transmitted by the rear face, the plasmon at the sample-metal interface can be excited by an incident beam on the inclined rear face according to a highly precise angle—corresponding to the agreement of the wave vector component parallel with the plasmon (Kretschmann configuration, cf. FIG. 2).

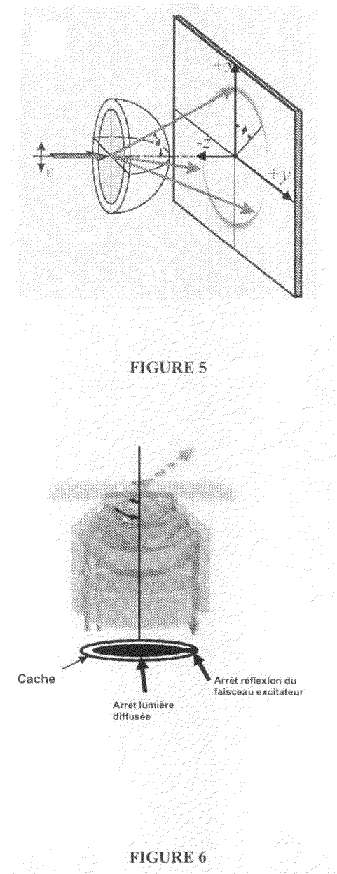

To this end, it is possible to use a configuration (and apparatus) similar to classic installations for excitations in evanescent waves (for reflectometry measurements of plasmon resonance or for those of total internal reflection fluorescence, cf. FIG. 6). It should be ensured that polarisation of the beam is of type p to excite the plasmon and place a cache to stop the reflected incident beam which lets through the rest of the cone of light coupled with the surface plasmon. It is also preferred that this cache stops all diffused light in the sample passing through the thin metallic film. It should be noted that when the incident beam has the right angle for exciting the plasmon, the intensity in the beam reflected is minimal. The parasitic light emanating from the excitation is thus reduced.

This type of excitation configuration can also be implemented with an installation using a hemispherical, hemicylindrical or triangular prism.

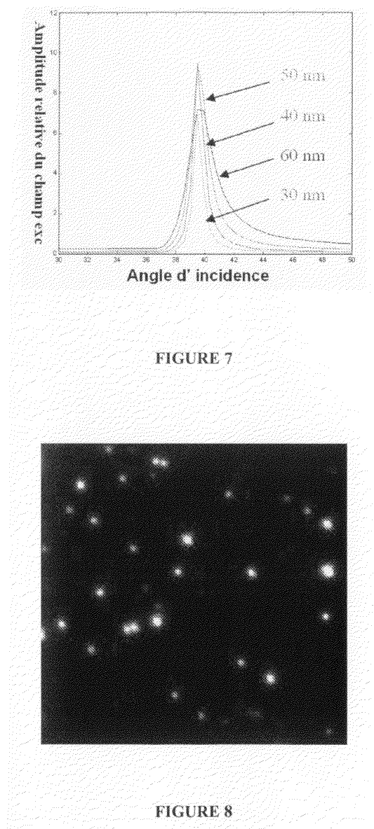

These excitation configurations correspond to preferred configurations as they have the advantage of lighting only those fluorophores located in the zone of interest (i.e. located in the evanescent wave) as in a standard TIRF system. Also, when the thickness of the metallic film is adjusted, the amplitude of this wave can be amplified relative to the incident beam of more than an order of magnitude, causing a significant increase in the signal relative to the standard TIRF (cf. FIG. 7).
B) The fluorophores can be also lit from the side of the sample (cf. FIG. 5)

This configuration is generally less interesting as there is no selectivity to excitation and the amplitude of the field near the surface is weaker (field practically zero at the level of the metal). However, spatial selection is made on emission as only those nanoparticles near the metallic surface can excite the plasmon. The diffusion of nanoparticles further from the surface (not coupled to the plasmon), even though strongly attenuated by the lead-through of the metallic layer, reaches the detector. The lobe of the diffused light is transmitted with a maximum angle less than that of the cone coming from coupling with the plasmon. It is also possible to filter this fluorescence using a cache in the form of a disc. It is possible, even in the case of excitation placed to the side of the sample, to fully filter the signal coming from the nanoparticles located far from the metal.

### Example 5

### Manufacture of Supports

The samples are made by thermal evaporation under vacuum. The thickness of the metallic layer is optimised for a given excitation wavelength. Other techniques can likewise be employed, this type of support not being difficult to make.

Silver or gold are candidates of choice and allow significant field amplifications in the visible spectrum. Other metals (aluminium, platinum, copper, . . . ) can also be used.

The method according to the invention was performed for example with gold nanoparticles of 20 nm in aqueous phase. These nanoparticles can be observed directly by the naked eye via microscope and their Brownian movement is visible.

### Example 6

Detection of Silver Nanoparticles of 20 nm in Diameter Deposited onto a Support

### Nanoparticles to be Detected

Individual silver nanoparticles, 20 nm in diameter.

### Support Used

Flat solid support made of glass (slide cover glass) covered with a silver film 50 nm in thickness. The nanoparticles to be detected are separated from the film metal by a layer of amorphous aluminium 15 nm in thickness.

### Excitation Wavelength of Nanoparticles

The deposited nanoparticles are excited by lighting of the upper surface of the support with a 100 W white halogen light whereof the spectrum comprises the wavelength related to the frequency of plasmon resonance of the nanoparticles silver used here (around 450 nm+/−30 nm).

### Detection (See FIG. 8)

The microscope used is an Olympus BH-2 or Nikon, with a digital wide-aperture immersion lens (1.49 ON). The camera used here is an EM-CCD Hamamatsu C-9100. The image obtained in FIG. 8 was binarised (black and white) for better display on paper in black and white.

Images of the same quality revealing the presence of individual silver nanoparticles of diameter 20 nm were obtained with the same support, though with lighting of the lower part of the support, with great care having to be taken to placing the mask allowing the reflected light originating from the light source to be eliminated via the lower surface of the solid support.

1. A method for detection and/or quantification of nanoparticles present on the upper surface of a flat solid support, said nanoparticles having a plasmon resonance, said solid support comprising a solid transparent support coated on its upper surface by a metallic film having a surface plasmon and the index of said transparent support being greater than that of the medium in which said nanoparticles are located, said method comprising:
a) lighting by the upper or lower surface of said support so as to illuminate the nanoparticles optionally present on the upper surface; and
b) detection and/or quantification of the light originating from the nanoparticles by the lower surface.

2. The method for detection and/or quantification of nanoparticles according to claim 1, wherein:
at step a) the lighting is generated by the upper surface; and
at step b) the detection and/or quantification of the light originating from the nanoparticles by the lower surface is done by releasing the direct light passing through the metallic film originating from the lighting,
or in that:
at step a) the lighting is generated by the lower surface; and
at step b) detection and/or quantification of the light originating from the nanoparticles by the lower surface is done by releasing the reflected light originating from the lighting.

3. The method for detection and/or quantification of nanoparticles according to claim 1, wherein the nanoparticles are metallic nanoparticles.

4. The method for detection and/or quantification of nanoparticles according to claim 3, wherein the nanoparticles are nanoparticles selected from nanoparticles of gold, silver, aluminium, platinum or copper.

5. The method for detection and/or quantification of nanoparticles according to claim 4, wherein the nanoparticles are nanoparticles selected from nanoparticles of gold or silver.

6. A manufacturing method according to claim 1, wherein said metallic film is selected from a film whereof the metal is that of the nanoparticles to be detected.

7. The manufacturing method according to claim 5, wherein said metallic film has a thickness of between 5 nm and 500 nm, preferably between 20 nm and 80 nm.

8. The method for detection and/or quantification of nanoparticles according to claim 1, wherein said nanoparticles likely to be present on the upper surface of the solid support to be detected and/or quantified are at a distance less than or equal to the excitation wavelength of said nanoparticles, preferably less than or equal to half of the excitation wavelength of said nanoparticles.

9. The method for detection and/or quantification of nanoparticles according to claim 1, wherein said nanoparticles likely to be present on the upper surface of the solid support to be detected and/or quantified are at a distance less than or equal to 500 nm if the light used for lighting is in the visible spectrum.

10. The method for detection and/or quantification of nanoparticles according to claim 1, wherein said nanoparticles likely to be present on the upper surface of the solid support to be detected and/or quantified are at a distance less than or equal to 500 nm, preferably less than or equal to 200 nm, of the upper surface of the solid support.

11. The method for detection and/or quantification of nanoparticles according to claim 1, wherein the metallic film located at the upper surface of the support is covered with a transparent film allowing the distance minimum between the nanoparticles and the metallic film to be adjusted.

12. The method for detection and/or quantification of nanoparticles according to claim 11, wherein said transparent film has a thickness less than or equal to 50 nm.

13. The method for detection and/or quantification of nanoparticles according to claim 1, wherein at step a) the lighting is done by the upper surface.

14. The method for detection of nanoparticles according to claim 1, wherein at step b) the light originating from the nanoparticles is transmitted to the lower surface via said support.

15. The method for detection and/or quantification of nanoparticles according to claim 1, wherein at step a) lighting by the upper or lower surface of said support is generated with a white light source or with polychromatic light whereof the excitation wavelengths contain at least one excitation wavelength related to the frequency of plasmon resonance of said nanoparticles.

16. The method for detection and/or quantification of nanoparticles according to claim 1, wherein at step a) the lighting by the upper or lower surface of said support is done with a monochromatic light source whereof the excitation wavelength is related to the frequency of plasmon resonance of said nanoparticles.

17. The method for detection and/or quantification of nanoparticles according to claim 1, wherein at step b) detection and/or quantification of the light originating from the nanoparticles on the lower surface is done by means of a microscope, where necessary coupled to a CCD camera.

18. The method for detection and/or quantification of nanoparticles according to claim 1, wherein the nanoparticles to be detected and/or quantified have a diameter less than or equal to 60 nm, preferably less than or equal to 20 nm.

19. The method for detection and/or quantification of nanoparticles according to claim 1, wherein the nanoparticles to be detected and/or quantified present colours associated with their plasmon resonance.

20. The method according to claim 1, wherein said support is a transparent solid support coated on its upper surface by a metallic film on which is fixed a probe compound capable of specifically recognising a target compound to be detected and/or quantified by means of or by the presence of nanoparticles.

21. A method for detection and/or quantification of a target compound, the method comprising:
a) at least one of (i) detecting and (ii) quantifying the target compound in a sample by a solid support, wherein said target compound is correlated to detection and/or quantification of nanoparticles;
b) lighting by the upper or lower surface of said support so as to illuminate the nanoparticles optionally present on the upper surface; and
c) detection and/or quantification of the light originating from the nanoparticles by the lower surface.

22. The method for detection and/or quantification according to claim 21, wherein the nanoparticles to be detected and/or quantified are used as specific marker of said target compound.

23. The method for detection and/or quantification according to claim 22, further comprising coating the nanoparticles to be detected and/or quantified with a compound capable of being bound specifically to the target compound.

24. The method for detection and/or quantification according to claim 21, further comprising selecting the target compound from the group of compounds constituted by nucleic acids, polypeptides, peptide-nucleic acids (PNA), lipopeptides, glycopeptides, carbohydrates, lipids, preferably nucleic acids, polypeptides or carbohydrates.

25. The method according to claim 21, further comprising making said flat solid support of glass.

26. A device for the detection and/or quantification of nanoparticles, the device comprising an upper surface of a flat solid support, said nanoparticles having a plasmon resonance, said solid support comprising a solid transparent support coated on its upper surface by a metallic film having a surface plasmon and the index of said transparent support being greater than that of the medium in which said nanoparticles are located, a light source allowing lighting of the upper surface or of the lower surface of said support and a system for detection and/or quantification of the light transmitted by the lower surface of said support, a system for eliminating or masking either:
reflected light originating from the light source when the lighting is generated by the lower surface of the solid support; or
direct light transmitted by the light source when the lighting is generated by the upper surface of the solid support.

27. The method according to claim 21, further comprising the steps of:
detection and/or quantification of a component present in a sample;
diagnosis;
biological imaging of systems confined to a few tens of nm, especially for the study of membranal transfers or biosensors; and
full-field imaging of nanoparticles, especially in diameter less than or equal to 20 nm, over a thickness less than or equal to 500 nm, preferably less than or equal to 200 nm.
